(19) United States
(12) Patent Application Publication
(10) Pub. No.: US 2024/0054360 A1
(43) Pub. Date: Feb. 15, 2024

(54) SIMILAR PATIENTS IDENTIFICATION METHOD AND SYSTEM BASED ON PATIENT REPRESENTATION IMAGE

(71) Applicant: ZHEJIANG LAB, Hangzhou (CN)

(72) Inventors: Tianshu ZHOU, Hangzhou (CN); Yifan JIANG, Hangzhou (CN); Jingsong LI, Hangzhou (CN); Yu TIAN, Hangzhou (CN); Ying ZHANG, Hangzhou (CN)

(21) Appl. No.: 18/358,051

(22) Filed: Jul. 25, 2023

(30) Foreign Application Priority Data
Aug. 11, 2022 (CN) 202210958286.9

(51) Int. Cl. G06N 5/022; G06T 5/10; G06T 11/20; G06V 10/75; G06V 10/74; G16H 10/60; G16H 50/70

(52) U.S. Cl. CPC G06N 5/022; G06T 5/10; G06T 11/20; G06V 10/751; G06V 10/761; G16H 10/60; G16H 50/70; G06T 2207/20052

(57) ABSTRACT

The present disclosure discloses a similar patients identification method and system based on a patient representation image. The method includes following steps: S1: building a healthcare knowledge graph: generating the healthcare knowledge graph by extracting entities and a relationship between the entities in a knowledge source; S2: building a healthcare knowledge graph space vector library; S3: building a patient's personal healthcare knowledge graph space vector data set; S4: drawing a patient's personal healthcare representation image; and S5: performing similar patients identification based on graph similarity calculation. The present disclosure builds a visual patient representation mode, so as to convert patient's healthcare data into a visual image, so that a doctor may intuitively feel a difference of different patients and similarity of similar patients.

### CROSS-REFERENCE TO RELATED APPLICATIONS

The present application claims priority to Chinese Patent Application No. 202210958286.9, filed on Aug. 11, 2022, the content of which is incorporated herein by reference in its entirety.

### TECHNICAL FIELD

The present disclosure relates to the technical field of medical information, in particular to a similar patients identification method and system based on a patient representation image.

### BACKGROUND

With widespread use of a medical information system, a large amount of clinical data has been generated. In clinical practice, doctors need to make diagnosis and treatment decisions for patients, often based on clinical guidelines or clinical experience. If patients similar to a current patient can be identified in the large amount of clinical data, and a similar patients cohort can be constructed and analyzed, it will help doctors make better diagnosis and treatment decisions for the current patient. At the same time, in the context of the reform of a medical insurance payment method, medical institutions are faced with a demand for cost control. For example, under a disease-related grouping payment mode, final grouping of the patients will not be determined until discharging from a hospital, thus affecting a medical insurance reimbursement ratio of the hospital. If the patient cohort similar to the current patient can be identified at an early stage, grouping situations, diagnosis and treatment paths, and cost of these similar patients can be analyzed, and accurate pre-grouping can thus be performed, the hospital is helped to improve the level of cost control, and optimize the clinical paths and diagnosis and treatment strategies.

Currently, there are some methods that use machine learning and deep learning to identify similar patients. However, on the one hand, these methods require a large amount of data annotations and training to improve the accuracy, and on the other hand, the methods based on machine learning and deep learning are usually black box models, which lack interpretation, and characteristics of the patients cannot be present to the doctor in an intuitive and understandable mode, and thus are difficult to understand and trust by the doctor.

Therefore, a similar patients identification method and system based on a patient representation image are proposed.

### SUMMARY

Aimed at solving shortcomings of the prior art, the present disclosure provides a similar patients identification method and system based on a patient representation image.

A technical solution adopted by the present disclosure is as follows:
a similar patients identification method based on a patient representation image, including following steps:
S1: building a healthcare knowledge graph: generating the healthcare knowledge graph by extracting entities and a relationship between the entities in a knowledge source;
S2: building a space vector library of the healthcare knowledge graph: converting all semantic meanings in the healthcare knowledge graph into space vectors and using an optimizer algorithm to perform training optimization based on a network search method to obtain the space vector library of the healthcare knowledge graph;
S3: building a space vector data set of a patient's personal healthcare knowledge graph: acquiring patient's personal healthcare data from a plurality of data sources, matching, extracting, converting and loading the patient's personal healthcare data, and mapping the data to the space vector library of the healthcare knowledge graph, and completing building of the space vector data set of the patient's personal healthcare knowledge graph;
S4: drawing a patient's personal healthcare representation image: reducing a dimensionality of the space vector data set of the patient's personal healthcare knowledge graph to a two-dimensional plane space through a principal component analysis method, so as to generate the patient's personal healthcare representation image; and
S5: performing similar patients identification based on image similarity calculation: calculating similarity between different patients by using an image similarity calculation method, and identifying similar patients from a patient's personal healthcare data set.

Furthermore, the knowledge source in S1 includes a related research literature, a clinical guideline and/or real-world data.

Furthermore, a data structure of the healthcare knowledge graph in S1 is designed as RDF triples conforming to an OWL language format specification; each triplet is used to represent entities and the relationship between entities, including two entities, a head entity and a tail entity, and the relationship between two entities; and the entities include demographic information, clinical diseases, symptoms, examinations, tests, drugs, and/or surgeries.

Furthermore, S2 specifically includes following sub-steps:
S21: using a healthcare standard term set as a data semantic identifier, and performing semantic identification on the entities and the relationship between the entities;
S22: using a semantic matching RESCAL model to convert all the semantic meanings into the space vectors, and obtaining the space vector library of the healthcare knowledge graph;
furthermore, S22 specifically includes following sub-steps:
S221: randomly initializing the space vectors;
S222: defining a scoring function;
S223: deducing an optimized loss function according to the scoring function; and
S224: training, through the optimizer algorithm, the initialized space vectors by using the optimized loss function and the network search method, and completing building of the space vector library of the healthcare knowledge graph.

Furthermore, the healthcare standard term set in S21 is built by adopting medical systematization naming-clinical terms, international classification of diseases, and/or a unified medical language system.

Furthermore, the data sources in S3 include clinical electronic medical records of medical institutions, personal health records and/or health questionnaire data; and the patient's personal healthcare data include basic personal information, demographic information, clinical diseases, symptoms, examinations, tests, drugs and/or surgeries.

Furthermore, S4 specifically includes following sub-steps:
S41: performing zero-mean on features of personal healthcare data of a random patient in the space vector data set of the patient's personal healthcare knowledge graph;
S42: calculating a covariance matrix of the space vector data set of the patient's personal healthcare knowledge graph;
S43: calculating feature values and feature vectors of the covariance matrix, sorting the feature values from large to small, and taking the feature vectors corresponding to the preset number of the feature values sorted from the front to form a conversion matrix;
S44: using the conversion matrix to reduce the dimensionality of the patient's individual healthcare data to obtain a two-dimensional plane space image after dimensionality reduction as the patient's personal healthcare representation image; and
S45: traversing steps S41 to S44 until patient's personal healthcare representation images of all patients are obtained.

Furthermore, S5 specifically includes following sub-steps:
S51: preprocessing the patient's personal healthcare representation image to obtain pixel points, and representing each pixel point by a gray value;
S52: performing discrete cosine transform (DCT) on the patient's personal healthcare representation image to obtain a DCT image;
S53: calculating a mean of the DCT image, comparing the mean with the gray value of each pixel point, and obtaining a hash value; and
S54: calculating different bits of the hash values of the different patient's personal healthcare representation images, setting a threshold value for determining whether patients are similar or dissimilar, and calculating a Hamming distance to obtain the similarity between the different patient's personal healthcare representation images, so as to identify similar patients from the space vector data set of the patient's personal healthcare knowledge image.

The present disclosure further provides a similar patients identification system based on a patient representation image, including:
a healthcare knowledge graph module, configured to extract entities and a relationship between the entities in a knowledge source to generate a healthcare knowledge graph;
a vector library module for the healthcare knowledge graph space, configured to convert all semantic meanings in the healthcare knowledge graph into space vectors and use an optimizer algorithm to perform training optimization based on a network search method to obtain a space vector library of the healthcare knowledge graph;
a space vector data set module for a patient's personal healthcare knowledge graph, configured to acquire patient's personal healthcare data from a plurality of data sources, to match, extract, convert and load the patient's personal healthcare data, and to map the data to the space vector library of the healthcare knowledge graph, and complete building of the space vector data set of the patient's personal healthcare knowledge graph;
a patient's personal healthcare representation image module, configured to reduce a dimensionality of the space vector data set of the patient's personal healthcare knowledge graph to a two-dimensional plane space through a principal component analysis method, so as to generate the patient's personal healthcare representation image; and
a similar patients identification module, configured to calculate similarity between different patients by using a graph similarity calculation method, and identify similar patients from a patient's personal healthcare data set.

The present disclosure has beneficial effects:
1. the present disclosure builds a visual patient representation mode, and converts patient's healthcare data into visual images, so that doctors can intuitively feel a difference of different patients and similarity of the similar patients. On this basis, the similar patients are identified, so the method is interpretable and doctors can understand and accept it more.
2. Based on a method of graph similarity calculation, the present disclosure performs similarity calculation on patient's representation images, so as to obtain the similarity between the patients, and a similar patients identification method without massive data training and annotations is built.

### BRIEF DESCRIPTION OF DRAWINGS

FIG. 1 is a schematic flow diagram of a similar patients identification method based on a patient representation image of the present disclosure.

FIG. 2 is a schematic structural diagram of a similar patients identification system based on a patient representation image of the present disclosure.

FIG. 3 is a schematic flow diagram of an embodiment.

### DESCRIPTION OF EMBODIMENTS

The following description of at least one exemplary embodiment is in fact illustrative only and never acts as any limitation on the present disclosure and its application or use. Based on the embodiments in the present disclosure, all other embodiments obtained by those ordinarily skilled in the art without creative labor fall within the scope of protection of the present disclosure.

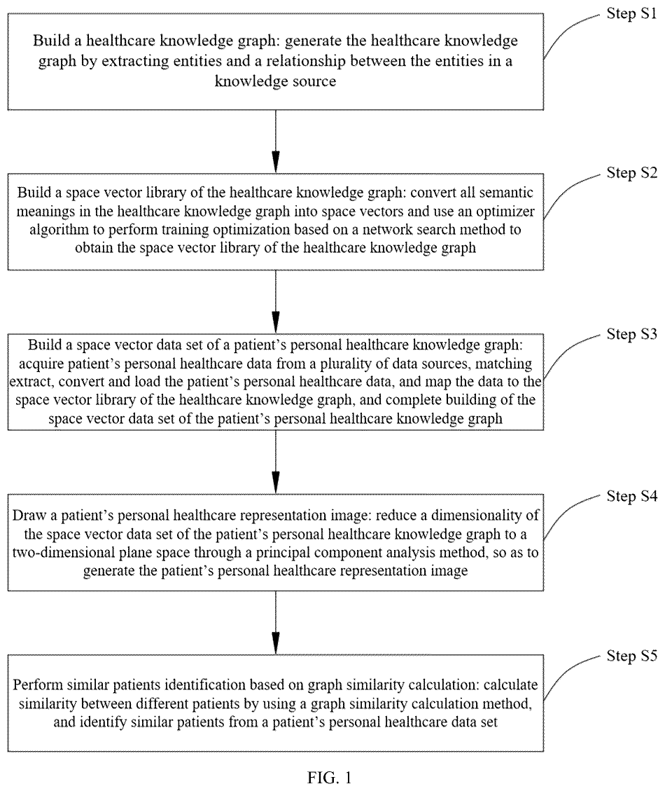

Referring to FIG. 1, a similar patients identification method based on a patient representation image includes following steps:
S1: a healthcare knowledge graph is built: the healthcare knowledge graph is generated by extracting entities and a relationship between the entities in a knowledge source;
the knowledge source includes a related research literature, a clinical guideline and/or real-world data; and
a data structure of the healthcare knowledge graph is designed as RDF triples conforming to an OWL language format specification; each triplet is used to represent entities and the relationship between entities, including two entities, a head entity and a tail entity, and the relationship between two entities, and the entities include demographic information, clinical diseases, symptoms, examinations, tests, drugs, and/or surgeries.
S2: a space vector library of the healthcare knowledge graph is built: all semantic meanings in the healthcare knowledge graph are converted into space vectors, and an optimizer algorithm is used to perform training optimization based on a network search method to obtain the space vector library of the healthcare knowledge graph;
S21: a healthcare standard term set is used as a data semantic identifier, and semantic identification is performed on the entities and the relationship between the entities;
the healthcare standard term set is built by adopting systematized nomenclature of medicine-clinical terms (SNOMED CTs), the international classification of diseases-10 (ICD-10), and/or a unified medical language system (UMLS).
S22: a semantic matching RESCAL model is used to convert all the semantic meanings into the space vectors, and the space vector library of the healthcare knowledge graph is obtained;
S221: random initializing is performed on the space vectors;
S222: a scoring function is defined;
S223: an optimized loss function is deduced according to the scoring function; and
S224: the initialized space vectors are trained, through the optimizer algorithm, by using the optimized loss function and the network search method, and building of the space vector library of the healthcare knowledge graph is completed.
S3: a space vector data set of a patient's personal healthcare knowledge graph is built: patient's personal healthcare data are acquired from a plurality of data sources, matching is performed on the patient's personal healthcare data, extracting, converting and loading are performed, then, the data is mapped to the space vector library of the healthcare knowledge graph, and building of the space vector data set of the patient's personal healthcare knowledge graph is completed; and
the data sources include clinical electronic medical records of medical institutions, personal health records and/or health questionnaire data; and the patient's personal healthcare data include basic personal information, demographic information, clinical diseases, symptoms, examinations, tests, drugs and/or surgeries.
S4: A patient's personal healthcare representation image is drawn: a dimensionality of the space vector data set of the patient's personal healthcare knowledge graph is reduced to a two-dimensional plane space through a principal component analysis method, so as to generate the patient's personal healthcare representation image;
S41: zero-mean is performed on features of personal healthcare data of a random patient in the space vector data set of the patient's personal healthcare knowledge graph;
S42: a covariance matrix of the space vector data set of the patient's personal healthcare knowledge graph is calculated;
S43: feature values and feature vectors of the covariance matrix are calculated, the feature values are sorted from large to small, and the feature vectors corresponding to the preset number of the feature values sorted from the front are taken to form a conversion matrix;
S44: the conversion matrix is used to reduce the dimensionality of the patient's personal healthcare data to obtain a two-dimensional plane space image after dimensionality reduction as the patient's personal healthcare representation image; and
S45: steps S41 to S44 are traversed until patient's personal healthcare representation images of all patients are obtained.
S5: Similar patients identification is performed based on image similarity calculation: similarity between different patients is calculated by using an image similarity calculation method, and similar patients are identified from a patient's personal healthcare data set;
S51: the patient's personal healthcare representation image is preprocessed to obtain pixel points, and each pixel point is represented by a gray value;
S52: discrete cosine transform (DCT) is performed on the patient's personal healthcare representation image to obtain a DCT image;
S53: a mean of the DCT image is calculated, and compared with the gray value of each pixel point, and a hash value is obtained; and
S54: different bits of the hash values of the different patient's personal healthcare representation images are calculated, a threshold value for determining whether patients are similar or dissimilar is set, and a Hamming distance is calculated to obtain the similarity between the different patient's personal healthcare representation images, so as to identify similar patients from the space vector data set of the patient's personal healthcare knowledge graph.

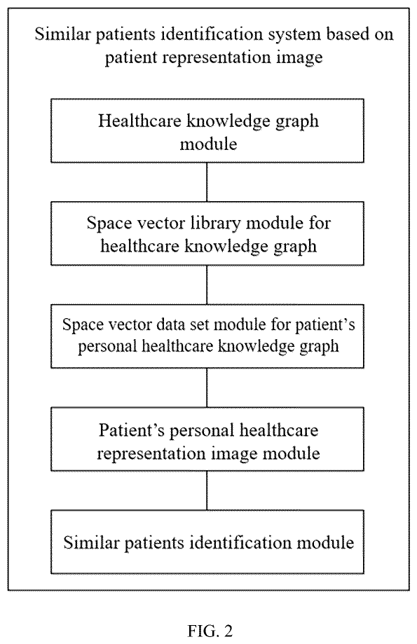

Referring to FIG. 2, a similar patients identification system based on a patient representation image includes:
a healthcare knowledge graph module, configured to extract entities and a relationship between the entities in a knowledge source to generate a healthcare knowledge graph;
a space vector library module for the healthcare knowledge graph, configured to convert all semantic meanings in the healthcare knowledge graph into space vectors and use an optimizer algorithm to perform training optimization based on a network search method to obtain a space vector library of the healthcare knowledge graph;
a space vector data set module for a patient's personal healthcare knowledge graph, configured to acquire patient's personal healthcare data from a plurality of data sources, to match, extract, convert and load the patient's personal healthcare data, and to map the data to the space vector library of the healthcare knowledge graph, and complete building of the space vector data set of the patient's personal healthcare knowledge graph;
a patient's personal healthcare representation image module, configured to reduce a dimensionality of the space vector data set of the patient's personal healthcare knowledge graph to a two-dimensional plane space through a principal component analysis method, so as to generate the patient's personal healthcare representation image; and
a similar patients identification module, configured to calculate similarity between different patients by using an image similarity calculation method, and identify similar patients from a patient's personal healthcare data set.

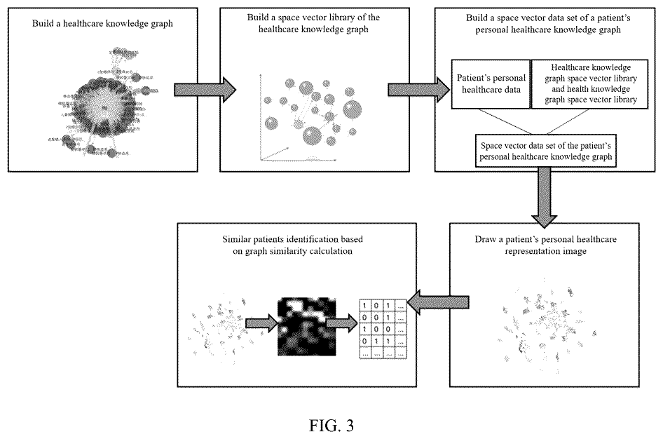

Embodiment: referring to FIG. 3, a similar patients identification method based on a patient representation image includes following steps:
S1: a healthcare knowledge graph is built: the healthcare knowledge graph is generated by extracting entities and a relationship between the entities in a knowledge source;
the knowledge source includes a related research literature, a clinical guideline and/or real-world data;
a natural language processing technology, generalization and summarization and other methods are used to extract knowledge from the knowledge source, and entities and a relationship between the entities are built, so that the healthcare knowledge graph is generated; and
a data structure of the healthcare knowledge graph is designed as resource description framework (RDF) triples conforming to a web ontology language (OWL) language format specification; each triplet is used to represent the entities and the relationship between entities, including two entities, a head entity and a tail entity, and the relationship between two entities, and the entities include demographic information, clinical diseases, symptoms, examinations, tests, drugs, and/or surgeries.
S2: a space vector library of the healthcare knowledge graph is built: all semantic meanings in the healthcare knowledge graph are converted into space vectors, and an optimizer algorithm is used to perform training optimization based on a network search method to obtain the space vector library of the healthcare knowledge graph;
S21: a healthcare standard term set is used as a data semantic identifier, and semantic identification is performed on the entities and the relationship between the entities; and
the healthcare standard term set is used as the data semantic identifier and used for identifying semantic meanings of the entities and the relationship between the entities and has uniqueness. The healthcare standard term set may be built by adopting systematized nomenclature of medicine-clinical terms (SNOMED CTs), the international classification of diseases-10 (ICD-10), and/or a unified medical language system (UMLS).
S22: A semantic matching RESCAL model is used to convert all the semantic meanings into the space vectors, and the space vector library of the healthcare knowledge graph is obtained; and
the semantic matching RESCAL model performs calculation of entity set relationship similarity by using latent semantic features in the space vectors, so as to judge a confidence of the triples.
S221: random initializing is performed on the space vectors;
S222: a scoring function is defined; and
the triple representing the entities and the relationship between the entities is set as (h, r, t), where h is the head entity, t is the tail entity, r is the relationship, the space vectors with dimensionalities being d are used, h and t respectively represent the head entity and the tail entity, and a matrix Mᵣ with a dimensionality being d*d is used to represent the relationship. The scoring function is:

fᵣ(h,t)=h^{T}Mᵣt

where, h^{T} is a transposed vector of h.
S223: an optimized loss function is deduced according to the scoring function;

Loss=max(0,−h^{T}Mᵣt+h′^{T}Mᵣt′+m)

where, m is an interval hyperparameter, h′ is a negative sample of h, and t′ is a negative sample of t.
S224: the initialized space vectors are trained, through the optimizer algorithm, by using the optimized loss function and the network search method, and building of the space vector library of the healthcare knowledge graph is completed.

When the optimized loss function is used to perform optimization training on healthcare knowledge graph space vectors, both positive and negative samples need to be provided at the same time. A score gap between the positive and negative samples should be widened as far as possible through the corresponding optimizer algorithm, so as to maximize a training loss. Generally speaking, in the case that training data only have positive samples, the negative samples may be generated by a negative sampling method. An Adam algorithm is used as an optimizer to perform training optimization based on a grid search method, so as to build the healthcare knowledge graph space vector library.
S3: a space vector data set of the patient's personal healthcare knowledge graph is built: patient's personal healthcare data are acquired from a plurality of data sources, matching is performed on the patient's personal healthcare data, extracting, converting and loading are performed, then, the data is mapped to the space vector library of the healthcare knowledge graph, and building of the space vector data set of the patient's personal healthcare knowledge graph is completed;
the data sources include clinical electronic medical records of medical institutions, personal health records and/or health questionnaire data; and
the patient's personal healthcare data include basic personal information, demographic information, clinical diseases, symptoms, examinations, tests, drugs and/or surgeries.

Terms adopted by the patient's personal healthcare knowledge graph space vector data set are kept consistent with the healthcare standard term set.

The patient's personal healthcare knowledge graph space vector data set is generally stored in a structural data mode, and mapping specifically refers to converting structural data into a form of the space vectors. Patient's personal relevant healthcare entities and the relationship between the entities are represented by the triples, and the entities and the relationship in the triples are all represented by the space vectors.
S4: a patient's personal healthcare representation image is drawn: a dimensionality of the space vector data set of the patient's personal healthcare knowledge graph is reduced to a two-dimensional plane space through a principal component analysis method, so as to generate the patient's personal healthcare representation image; and
PCA is a common statistical analysis method for dimensionality reduction of high-dimensional data, its principle is to convert and map high-dimensional data into low-dimensional space data in a linear projection mode, and its goal is to find a projection method that maximizes a variance.

A data set of a certain patient in the patient's personal healthcare knowledge graph space vector data set is set as X={x₁, x₂, . . . , xₘm}, personal healthcare data xᵢ of each patient is a space vector with a dimensionality as d, the dimensionality is reduced to a dimensionality of a low-dimensional space for n, and a value of n is 2 here.
S41: Zero-mean is performed on features of personal healthcare data of a random patient in the space vector data set of the patient's personal healthcare knowledge graph.

Zero-mean is performed on features of the patient's personal healthcare data, that is, a mean of each feature in the patient's personal healthcare knowledge graph space vector data set is subtracted from the feature of the personal healthcare data of each patient. For a j^{th} feature of the personal healthcare data xᵢ of an i^{th} patient:

xᵢ^{j}=xᵢ^{j}−μⱼ

where, μⱼ is the mean of the j^{th} feature in the patient's personal healthcare knowledge graph space vector data set, that is μⱼ=1/mΣₖ₌₁^{m}xₖ^{j}.
S42: A covariance matrix: Σ=XX^{T} of the space vector data set of the patient's personal healthcare knowledge graph is calculated;
S43: feature values and feature vectors of the covariance matrix are calculated, the feature values are sorted from large to small, and the feature vectors corresponding to the preset number of the feature values sorted from the front are taken to form a conversion matrix;
feature vectors corresponding to first n feature values are taken to form the conversion matrix U;
S44: the conversion matrix is used to reduce the dimensionality of the patient's personal healthcare data to obtain a two-dimensional plane space image after dimensionality reduction as the patient's personal healthcare representation image;
the patient's personal healthcare data are converted to a new low-dimensional space, a data set after dimensionality reduction is set as Y={y₁, y₂, . . . , yₘ}, so yᵢ=U^{T}xᵢ; and
S45: steps S41 to S44 are traversed until patient's personal healthcare representation images of all patients are obtained.
S5: Similar patients identification is performed based on graph similarity calculation: similarity between different patients is calculated by using a graph similarity calculation method, and similar patients are identified from a patient's personal healthcare data set.

Similarity calculation is performed on the patient's personal healthcare representation image based on a pHash algorithm. The pHash algorithm, also known as a perceptual hash algorithm, processes the image to generate a fingerprint, and then the fingerprints between different images are compared so as to calculate the similarity of the images.
S51: The patient's personal healthcare representation image is preprocessed to obtain pixel points, and each pixel point is represented by a gray value; and
each patient's personal healthcare representation image is preprocessed, all the patient's personal healthcare representation images are reduced to a 32*32 size, with a total of 1024 pixels, then graying processing is performed on each pixel point, and each pixel point is represented by the gray value.
S52: Discrete cosine transform (DCT) is performed on the patient's personal healthcare representation image to obtain a DCT image;
DCT is performed on the patient's personal healthcare representation image to change the patient's personal healthcare representation image from a pixel domain to a frequency domain. DCT, also known as discrete cosine transform, is a transformation method evolved based on discrete Fourier transform. Based on the discrete Fourier transform, the Fourier transform for a real even function only includes a real cosine term, thus forming the DCT for a real number domain. A formula of two-dimensional DCT is as follows:

$$F ( u , v ) = c ( u ) c ( v ) {\sum }_{i}^{N - 1} {\sum }_{j}^{N - 1} f ( i , j ) cos [ \frac{( 2 i + 1 ) π}{2 N} u ] cos [ \frac{( 2 j + 1 ) π}{2 N} v ]$$

where, f(i, j) is an element of a space two-dimensional vector, F(u, v) is an element of a transformation coefficient array, N is a number of time domain sequence points, and c(u) and c(v) are coefficients:

$$c ( u ) = \{ \begin{matrix}\sqrt{1 / N} & u = 0 \\ \sqrt{2 / N} & u \neq 0\end{matrix} \\ c ( v ) = \{ \begin{matrix}\sqrt{1 / N} & v = 0 \\ \sqrt{2 / N} & v \neq 0\end{matrix}$$

after DCT, the DCT image is obtained, and a size is 32*32.
S53: A mean of the DCT image is calculated, and compared with the gray value of each pixel point, and a hash value is obtained; and
then binaryzation is performed, that is, a hash value is calculated. Firstly, the mean of the DCT image is determined, then, each pixel point is compared with the mean, if the pixel point is greater than or equal to the mean, a value is 1, otherwise, the value is 0, and therefore the hash value of 1024 bits is obtained.
S54: Different bits of the hash values of the different patient's personal healthcare representation images are calculated, a threshold value for determining whether patients are similar or dissimilar is set, and a Hamming distance is calculated to obtain the similarity between the different patient's personal healthcare representation images, so as to identify the similar patients from the space vector data set of the patient's personal healthcare knowledge graph.

The above embodiments are only preferred embodiments of the present disclosure and are not intended to limit the present disclosure, and for those skilled in the art, the present disclosure may have various changes and variations. Any modifications, equivalent substitutions, improvement, etc. made within the spirit and principles of the present disclosure shall be included in the scope of protection of the present disclosure.

1. A similar patients identification method based on a patient representation image, comprising steps of:
step S1: building a healthcare knowledge graph: generating the healthcare knowledge graph by extracting entities and a relationship between the entities in a knowledge source;
wherein a data structure of the healthcare knowledge graph is designed as RDF triples conforming to an OWL language format specification; each triplet is used to represent entities and the relationship between the entities, comprising two entities, a head entity and a tail entity, and the relationship between the two entities; and the head entity and the tail entity comprise demographic information, clinical diseases, symptoms, examinations, tests, drugs, and/or surgeries;
step S2: building a space vector library of the healthcare knowledge graph: converting all semantic meanings in the healthcare knowledge graph into space vectors and using an optimizer algorithm to perform training optimization based on a network search method to obtain the space vector library of the healthcare knowledge graph;
step S21: using a healthcare standard term set as a data semantic identifier, and performing semantic identification on the entities and the relationship between the entities;
step S22: using a semantic matching RESCAL model to convert all the semantic meanings into the space vectors, and obtaining the space vector library of the healthcare knowledge graph;
step S221: randomly initializing the space vectors;
step S222: defining a scoring function;
step S223: deducing an optimized loss function according to the scoring function;
step S224: training, through the optimizer algorithm, the initialized space vectors by using the optimized loss function and the network search method, and completing building of the space vector library of the healthcare knowledge graph;
step S3: building a space vector data set of a patient's personal healthcare knowledge graph: acquiring patient's personal healthcare data from a plurality of data sources, matching, extracting, converting and loading the patient's personal healthcare data, and mapping the data to the space vector library of the healthcare knowledge graph, and completing building of the space vector data set of the patient's personal healthcare knowledge graph;
step S4: drawing a patient's personal healthcare representation image: reducing a dimensionality of the space vector data set of the patient's personal healthcare knowledge graph to a two-dimensional plane space through a principal component analysis method, so as to generate the patient's personal healthcare representation image;
step S41: performing zero-mean on features of personal healthcare data of a random patient in the space vector data set of the patient's personal healthcare knowledge graph;
step S42: calculating a covariance matrix of the space vector data set of the patient's personal healthcare knowledge graph;
step S43: calculating feature values and feature vectors of the covariance matrix, sorting the feature values from large to small, and taking the feature vectors corresponding to the preset number of the feature values sorted from the front to form a conversion matrix;
step S44: using the conversion matrix to reduce the dimensionality of the patient's personal healthcare data to obtain a two-dimensional plane space image after dimensionality reduction as the patient's personal healthcare representation image;
step S45: traversing step S41 to step S44 until patient's personal healthcare representation images of all patients are obtained;
step S5: performing similar patients identification based on graph similarity calculation: calculating similarity between different patients by using a graph similarity calculation method, and identifying similar patients from a patient's personal healthcare data set;
step S51: preprocessing the patient's personal healthcare representation image to obtain pixel points, and representing each pixel point by a gray value;
step S52: performing discrete cosine transform (DCT) on the patient's personal healthcare representation image to obtain a DCT image;
step S53: calculating a mean of the DCT image, comparing the mean with the gray value of each pixel point, and obtaining a hash value; and
step S54: calculating different bits of the hash values of the different patient's personal healthcare representation images, setting a threshold value for determining whether patients are similar or dissimilar, and calculating a Hamming distance to obtain the similarity between the different patient's personal healthcare representation images, so as to identify the similar patients from the space vector data set of the patient's personal healthcare knowledge graph.

2. The similar patients identification method based on a patient representation image according to claim 1, wherein the knowledge source in step S1 comprises a literature, a clinical guideline and/or real-world data.

3. The similar patients identification method based on a patient representation image according to claim 1, wherein the healthcare standard term set in step S21 is built by adopting systematized nomenclature of medicine-clinical terms, international classification of diseases, and/or a unified medical language system.

4. The similar patients identification method based on a patient representation image according to claim 1, wherein the data sources in step S3 comprise clinical electronic medical records of medical institutions, personal health records and/or health questionnaire data; and the patient's personal healthcare data comprise basic personal information, demographic information, clinical diseases, symptoms, examinations, tests, drugs and/or surgeries.

5. A system configured to implement the similar patients identification method based on a patient representation image according to claim 1, comprising:
a healthcare knowledge graph module, configured to extract entities and a relationship between the entities in a knowledge source to generate a healthcare knowledge graph;
a space vector library module for the healthcare knowledge graph, configured to convert all semantic meanings in the healthcare knowledge graph into space vectors and use an optimizer algorithm to perform training optimization based on a network search method to obtain a space vector library of the healthcare knowledge graph;
a space vector data set module for a patient's personal healthcare knowledge graph, configured to acquire patient's personal healthcare data from a plurality of data sources, to match, extract, convert and load the patient's personal healthcare data, and to map the data to the space vector library of the healthcare knowledge graph, and complete building of the space vector data set of the patient's personal healthcare knowledge graph;
a patient's personal healthcare representation image module, configured to reduce a dimensionality of the space vector data set of the patient's personal healthcare knowledge graph to a two-dimensional plane space through a principal component analysis method, so as to generate the patient's personal healthcare representation image; and
a similar patients identification module, configured to calculate similarity between different patients by using a graph similarity calculation method, and identify similar patients from a patient's personal healthcare data set.
